(19) United States
(12) Patent Application Publication
(10) Pub. No.: US 2026/0146234 A1
(43) Pub. Date: May 28, 2026

(54) 3D-PRINTED SKIN MODEL USING PERSONALIZED DECELLULARIZED ECM

(71) Applicants: Ewha University-Industry Collaboration Foundation, Seoul (KR); POSTECH RESEARCH AND BUSINESS DEVELOPMENT FOUNDATION, Pohang-si (KR)

(72) Inventors: Junmin LEE, Pohang-si (KR); Bo Young PARK, Seoul (KR); Raehui KANG, Pohang-si (KR)

(73) Assignees: Ewha University-Industry Collaboration Foundation, Seoul (KR); POSTECH RESEARCH AND BUSINESS DEVELOPMENT FOUNDATION, Pohang-si (KR)

(21) Appl. No.: 19/401,991

(22) Filed: Nov. 26, 2025

(30) Foreign Application Priority Data
Nov. 27, 2024 (KR) 10-2024-0172521

(51) Int. Cl. C12N 5/077; B33Y 70/00; B33Y 80/00; C12N 5/00; C12N 5/071; C12N 9/64; C12N 9/76

(52) U.S. Cl. CPC C12N 5/0656; B33Y 70/00; B33Y 80/00; C12N 5/0018; C12N 5/0629; C12N 9/6427; C12N 9/6489; C12N 2500/25; C12N 2500/32; C12N 2500/34; C12N 2500/84; C12N 2501/11; C12N 2501/119; C12N 2501/39; C12N 2501/999; C12Y 304/21004; C12Y 304/24003; C12Y 304/24004

(57) ABSTRACT

The present invention relates to a method for producing a skin tissue model, comprising: (a) isolating and culturing fibroblasts and keratinocytes from skin tissue; (b) preparing a bioink composition for a dermal layer and a bioink composition for an epidermal layer, the bioink composition containing the isolated cells; and (c) producing a skin tissue model using the bioink composition for a dermal layer and the bioink composition for an epidermal layer.

### CROSS-REFERENCE TO RELATED APPLICATIONS

This application claims priority to Korean Patent Application No. 10-2024-0172521 filed on Nov. 27, 2024, the entire disclosure of which is incorporated herein by reference.

### TECHNICAL FIELD

The present disclosure relates to a method for producing an artificial skin tissue using bioink and an artificial skin and/or a skin structure produced therefrom, and more particularly, to a 3D-printed skin model using personalized decellularized extracellular matrix (ECM).

### BACKGROUND ART

In the fields of tissue engineering and regenerative medicine, there is an increasing demand to produce artificial tissues with high physiological similarity to the human body by simulating the microenvironment inside the human body using cell-friendly biomaterials. These techniques play an important role in replacing animal experiments or in producing transplantable artificial tissues.

The skin, the largest organ of the human body, performs a function of preventing the loss of body fluids, preventing the penetration of external harmful substances and microorganisms, and protecting the body from physical and chemical stimuli. In addition, the skin is an organ in which symptoms of various diseases appear, and the condition of the patient may be diagnosed and predicted through symptoms such as skin aging, dermatitis, urticaria, and allergic reactions.

For patients with severe skin damage due to severe burns, trauma, epithelial cancer resection, and skin diseases, it is necessary to form a protective film capable of preventing infection of the lost area and fluid loss. Further, it is important to prevent scarring in the wound area and to prevent serious contraction that may occur during the healing process.

Methods for regenerating damaged skin include autograft, homograft (or allograft), heterograft (or xenograft), etc. The autograft is a method of transplanting the patient's own skin, which is considered the most ideal method, but when the damaged area is extensive, the availability of donor tissue is limited, and additional wounds are created at the extraction site. In addition, in the case of autologous keratinocyte sheets, the reference wound is excised, a dermal substitute is transplanted, and treatment is performed using the keratinocyte sheets, and thus this approach prolongs the wound healing and regeneration period and often requires multiple surgical procedures, imposing psychological and economic burdens on the patient.

In order to solve this problem, the development of patient-specific bio-artificial skin without rejection response has become important in regenerative medicine recently. The recently developed tissue engineering three-dimensional skin model is a structure that mimics the original appearance of the skin including both the epidermal and dermal layers, and the importance of this technology is further emphasized. However, there is a limitation in that it is not yet possible to completely simulate the microstructure between the epidermis and the dermis, and it is difficult to optimize for individual subjects.

In order to overcome these limitations, it is necessary to develop a patient-specific skin tissue model or a skin structure that has excellent cell viability and viability retention and has fewer side effects and a fast recovery rate through a relatively simple and efficient process.

### DISCLOSURE

### Technical Problem

An object of the present disclosure is to provide a method for producing a skin tissue model.

Another object of the present disclosure is to provide a skin tissue model produced according to the method for producing the skin tissue model.

Still another object of the present disclosure is to provide a skin tissue graft material produced according to the method for producing the skin tissue model.

Still another object of the present disclosure is to provide a method for treating a skin wound or a method for regenerating the skin, the method comprising transplanting the skin tissue model or the skin tissue graft material to an individual.

### Technical Solution

Terms used in the present application are only used to describe specific embodiments and are not intended to limit the present disclosure. Singular expressions shall include plural expressions unless the context clearly indicates otherwise.

Various modifications may be applied to the embodiments described below. It should be understood that the embodiments described below are not intended to be limited to the embodiments, but include all changes, equivalents, and substitutes thereof.

Terms used in the present application are only used to describe specific embodiments and are not intended to limit the present disclosure. Singular expressions shall include plural expressions unless the context clearly indicates otherwise. In the present specification, terms such as “comprise” or “have” are intended to designate the presence of features, numbers, steps, operations, components, parts, or combinations thereof described in the specification and it should not be understood as precluding the possibility of the presence or addition of one or more other features, numbers, steps, operations, components, parts, or combinations thereof.

In addition, terms including ordinal numbers such as first, second, and the like, which will be used below, may be used to describe various components, but the components are not limited by the terms. The terms are used only for the purpose of distinguishing one component from another component. For example, the first element could be termed the second element, and, similarly, the second element could be termed the first element, without departing from the scope of the present disclosure.

Further, when a component is referred to as being “formed” or “laminated” on another component, it may be directly attached to the entire surface or one surface of the other component and formed or laminated, but it should be understood that other components may further exist in between.

Unless otherwise defined, all terms used herein, including technical or scientific terms, have the same meanings as those generally understood by those skilled in the art to which the present disclosure pertains. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

Hereinafter, a method for producing a skin tissue model according to an embodiment of the present disclosure and a skin tissue model produced according to the method will be described.

### Method for Producing Skin Tissue Model

In one general aspect, the present disclosure provides a method for producing a skin tissue model, comprising:
(a) isolating and culturing fibroblasts and keratinocytes from skin tissue;
(b) preparing a bioink composition for a dermal layer and a bioink composition for an epidermal layer, the bioink composition containing the isolated cells; and
(c) producing a skin tissue model using the bioink composition for a dermal layer and the bioink composition for an epidermal layer.

The method for producing a skin tissue model according to an embodiment of the present disclosure will be described in detail for each step.

(a) Step of Isolating and Culturing Fibroblasts and Keratinocytes from Skin Tissue

The step (a) may comprise a step of preparing a skin tissue from which cells are to be isolated.

The skin tissue may be a skin tissue isolated from a human.

In the cell isolation method according to an embodiment of the present disclosure, human skin tissue is used. Human skin tissues include the epidermal layer and the dermal layer.

In an embodiment, Step (a) may comprise washing the prepared skin tissue. In the washing step, the skin tissue is washed at least three times with a physiological buffer (e.g., phosphate buffered saline solution) to remove coagulated blood, hair, and fat.

In an embodiment, when the fat layer is removed, since fat tissue may be embedded in the dermis, a process of removing even fine adipose tissue using surgical precision scissors may be additionally performed.

In an embodiment, Step (a) may be performed by the following method:
(a-1) isolating epidermis and dermis from skin tissue using a solution containing Dispase-II;
(a-2) isolating keratinocytes from the isolated epidermis using a solution containing trypsin-ethylenediaminetetraacetic acid (EDTA) (trypsin-EDTA), and culturing the isolated keratinocytes in a keratinocyte culture medium; and
(a-3) isolating the fibroblasts from the dermis using a solution containing Collagenase-I and culturing the isolated fibroblasts in a fibroblast culture medium.

Steps (a-2) and (a-3) may be performed individually, and are not necessarily performed sequentially. In other words, Steps (a-2) and (a-3) may be performed simultaneously and need not be performed sequentially.

Specifically, in Step (a-1), the solution containing Dispase-II may contain a physiological buffer solution, and specifically, the buffer solution may be phosphate buffered saline.

The solution containing Dispase-II is a solution for isolating the epidermis and dermis from skin tissue.

The concentration of the dispase-II solution may be 2 to 5 UI/mL, for example, the lower limit may be 2.1 UI/mL or more, 2.2 UI/mL or more, 2.3 UI/mL or more, or 2.2 UI/mL or more, and the upper limit may be 4.8 UI/mL or less, 4.6 UI/mL or less, 4.4 UI/mL or less, 4.2 UI/mL or less, 4.0 UI/mL or less, 3.5 UI/mL or less, 3.0 UI/mL or less, or 2.5 UI/mL or less.

In an embodiment, in Step (a-1), the dispase-II solution may be treated for 5 to 20 hours, preferably for 10 to 20 hours, more preferably for 12 to 20 hours, and even more preferably for 15 to 19 hours.

In an embodiment, the dispase-II solution in Step (a-1) may be treated at 2 to 6° C., preferably at a temperature of about 4° C. When the epidermis of the tissue is not well isolated, the dispase-II solution may be further treated at a temperature of 37° C.

In an embodiment, in Step (a-1), after the dispase-II treatment is completed, the epidermis and the dermis may be isolated by forceps. Any remaining portion that is not isolated after further processing may be treated using a blade so that the epidermis may be isolated from the dermis.

In an embodiment, in Step (a-2), after removing the dispase-II solution by washing the isolated epidermis with PBS, a process of isolating keratinocytes from the isolated epidermis using a solution containing trypsin-ethylenediaminetetraacetic acid (EDTA) (trypsin-EDTA) may be performed.

In an embodiment, in Step (a-2), a solution containing 0.02 to 0.3% trypsin-ethylenediaminetetraacetic acid (trypsin-EDTA) may be used, and preferably, a solution containing 0.03 to 0.08%, more preferably 0.04 to 0.06% trypsin-ethylenediaminetetraacetic acid (trypsin-EDTA) may be used.

In an embodiment, in Step (a-2), the isolation of keratinocytes from the epidermis may be performed at a temperature of 25 to 40° C., preferably 30 to 39° C., and more preferably 35 to 38° C.

In an embodiment, in Step (a-2), the isolation of keratinocytes from the epidermis may be performed for 10 to 60 minutes, preferably for 20 to 40 minutes.

In an embodiment, the keratinocyte culture medium of Step (a-2) may be a keratinocyte basal medium (KBM) comprising penicillin/streptomycin, glutamine, epidermal growth factor (EGF), insulin, hydrocortisone, gentamicin/amphotericin-B, epinephrine, and transferrin. The keratinocyte culture medium may be a medium that does not contain calcium, but is not limited thereto.

In an embodiment, the keratinocyte culture medium may be a KBM medium comprising 0.5 to 2% (v/v) penicillin/streptomycin, 0.05 to 0.2% (v/v) glutamine, 0.2 to 0.8% (v/v) EGF, 0.05 to 0.2% (v/v) insulin, 0.05 to 0.2% (v/v) hydrocortisone, 0.05 to 0.2% (v/v) gentamicin/amphotericin-B, 0.02 to 0.1% (v/v) epinephrine, and 0.05 to 0.2% (v/v) transferrin.

Preferably, the keratinocyte culture medium may be a KBM medium comprising 1% (v/v) penicillin/streptomycin, 0.1% (v/v) glutamine, 0.4% (v/v) EGF, 0.1% (v/v) insulin, 0.1% (v/v) hydrocortisone, 0.1% (v/v) gentamicin/amphotericin-B, 0.05% (v/v) epinephrine, and 0.1% (v/v) transferrin.

In an embodiment, before Step (a-3), preparing a collagenase-I solution may be performed. The collagenase-I solution may be prepared as a 0.05 to 0.08% solution based on cell culture medium and may be prepared by adding 80 to 120 μl fibroblast growth factors per 1 mL.

In an embodiment, in Step (a-3), the collagenase-I solution may be a solution containing a keratinocyte culture medium described in the present disclosure, and does not contain a keratinocyte growth factor. In addition, the collagenase-I solution may comprise 80 to 120 μl fibroblast growth factors per 1 mL.

In an embodiment, in Step (a-3), 0.03 to 0.2% collagenase-I solution may be used, preferably 0.05 to 0.09%, more preferably 0.06 to 0.08% collagenase-I solution may be used, and most preferably about 0.07% collagenase-I solution may be used. In an embodiment, 0.16% of collagenase at 260 U/mL may be used.

In an embodiment, in Step (a-3), a collagenase-I solution having a temperature of 35 to 38° C., preferably 36 to 37° C. may be used.

In an embodiment, in Step (a-3), the isolation of the fibroblasts from the dermis may be performed at a temperature of 25 to 40° C., preferably 30 to 39° C., and more preferably 35 to 38° C.

In an embodiment, in Step (a-3), the isolation of the fibroblasts from the dermis may be performed for 10 to 120 minutes, preferably for 15 to 100 minutes, and more preferably for 20 to 70 minutes.

In an embodiment, the fibroblast culture medium of Step (a-3) may comprise fetal bovine serum, D-glucose, penicillin/streptomycin, L-glutamine, and fibroblast growth factors.

In an embodiment, the fibroblast culture medium may comprise 10% fetal bovine serum, 2 to 9 mg/mL D-glucose, 2 to 10 mL of penicillin/streptomycin (10,000 UI/mL), 2 to 8 mM L-glutamine, and 0.05 to 1 μg/mL fibroblast growth factor, and does not comprise pyruvic acid and hydroxyethylpiperazine ethanesulfonic acid.

### (b) Step of Preparing BioInk Composition for Dermal Layer and BioInk Composition for Epidermal Layer Including Isolated Cells

In an embodiment, the bioink composition for a dermal layer of Step (b) may comprise fibroblasts isolated from the skin tissue; dermis decellularized extracellular matrix (dermis dECM); and a bioink containing gelatin methacryloyl (GelMA) and alginate.

The bioink contained in the bioink composition for a dermal layer may comprise 3 to 15 w/v % gelatin methacryloyl (GelMA) and 0.5 to 4 w/v % alginate, preferably 4 to 13 w/v % gelatin methacryloyl (GelMA) and 0.6 to 3 w/v % alginate, and more preferably 4 to 12 w/v % gelatin methacryloyl (GelMA) and 0.8 to 2.5 w/v % alginate.

The bioink composition for a dermal layer may comprise the dermis decellularized extracellular matrix at a concentration of 0.5 to 2 w/v %, preferably 0.6 to 0.18 w/v %, and more preferably 0.8 to 0.15 w/v %.

The bioink composition for a dermal layer may be prepared by mixing the dermis dECM and the bioink containing gelatin methacryloyl (GelMA) and alginate in a volume ratio of 1:1 to 1:8, preferably in a volume ratio of 1:2 to 1:6, and more preferably in a volume ratio of 1:2, 1:3 or 1:4, and most preferably in a volume ratio of 1:4.

The bioink composition for a dermal layer may comprise fibroblasts at a concentration of 1×10⁶ to 1×10⁷ cells/ml, and for example, the lower limit may be 1.5×10⁶ cells/ml or more, 2×10⁶ cells/ml or more, 2.5×10⁶ cells/ml or more, 3×10⁶ cells/ml or more, 3.5×10⁶ cells/ml or more, 4×10⁶ cells/ml or more, 4.5×10⁶ cells/ml or more, or 5×10⁶ cells/ml or more, and the upper limit may be 9.5×10⁶ cells/ml or less, 9×10⁶ cells/ml or less, 8.8×10⁶ cells/ml or less, 8.6×10⁶ cells/ml or less, 8.4×10⁶ cells/ml or less, 8.2×10⁶ cells/ml or less, or 8×10⁶ cells/ml or less.

The dermis dECM may comprise at least one selected from the group consisting of collagens, proteoglycans, ECM glycoproteins, ECM-affiliated proteins, and ECM regulators.

Specifically, the dermis dECM may comprise collagens such as CoL6A1, CoL3A1, CoL1A1, CoL1A2; proteoglycans such as DCN, LUM, OGN, and PRELP; ECM glycoproteins such as DPT, POSTN, and ELN; ECM regulators such as LOX, CTSG, PRSS3; and ECM-affiliated proteins such as LGALS3.

The dermis dECM may comprise at least one of CoL6A1, CoL3A1, CoL1A1, CoL1A2, DCN, LUM, OGN, PRELP, DPT, POSTN, ELN, LOX, CTSG, PRSS3 and LGALS3.

The dermis dECM may be obtained by decellularizing dermal tissue isolated from human skin tissue.

In an embodiment, the bioink composition for an epidermal layer of Step (b) may comprise keratinocytes isolated from the skin tissue; gelatin methacryloyl (GelMA); and keratin-alginate.

The bioink composition for an epidermal layer may comprise 2 to 15 w/v % gelatin methacryloyl (GelMA) and 0.5 to 5 w/v % keratin-alginate, preferably 3 to 13 w/v % gelatin methacryloyl (GelMA) and 1 to 4 w/v % keratin-alginate, and more preferably 4 to 12 w/v % gelatin methacryloyl (GelMA) and 1 to 3 w/v % keratin-alginate.

The bioink composition for an epidermal layer may comprise keratinocytes at a concentration of 1×10⁶ to 4×10⁶ cells/ml, and the lower limit may be 1.2×10⁶ cells/ml or more, 1.4×10⁶ cells/ml or more, 1.6×10⁶ cells/ml or more, or 1.8×10⁶ cells/ml or more, and the upper limit may be 3.8×10⁶ cells/ml or less, 3.6×10⁶ cells/ml or less, 3.4×10⁶ cells/ml or less, 3.2×10⁶ cells/ml or less, 3×10⁶ cells/ml or less, 2.8×10⁶ cells/ml or less, 2.6×10⁶ cells/ml or less, 2.4×10⁶ cells/ml or less, or 2.2×10⁶ cells/ml or less.

### (c) Step of Producing Skin Tissue Model Using Bioink Composition for Epidermal Layer and Bioink Composition for Dermal Layer

Step (c) may comprise:
(c-1) forming a dermal layer by three-dimensionally printing the bioink composition for a dermal layer containing the fibroblasts isolated from skin tissue; and
(c-2) forming an epidermal layer on the dermal layer by three-dimensionally printing the bioink composition for an epidermal layer containing the keratinocytes isolated from skin tissue.

In an embodiment, Step (c-2) may be performed 2 to 4 times, so that the 2 to 4 epidermal layers may be laminated. In other words, the epidermal layer of the skin tissue model produced according to the present disclosure may be in the form of being laminated in multiple layers.

In the present disclosure, the dermal layer may have a thickness of 500 to 8000 μm, for example, the lower limit may be 550 μm or more, 600 μm or more, 650 μm or more, 700 μm or more, 750 μm or more, 800 μm or more, 850 μm or more, 900 μm or more, 950 μm or more, or 1000 μm or more, and the upper limit may be 7500 μm or less, 7000 μm or less, 6500 μm or less, 6000 μm or less, 5500 μm or less, 5000 μm or less, 4500 μm or less, or 4000 μm or less. The dermal layer of the present disclosure may be produced to the thickness of an actual skin dermal layer.

In the present disclosure, a thickness of a single layer of the epidermal layer may be 15 to 500 μm, the lower limit may be 20 μm or more, 25 μm or more, 30 μm or more, 35 μm or more, 40 μm or more, or 45 μm or more, and the upper limit may be 450 μm or less, 400 μm or less, 350 μm or less, 300 μm or less, 250 μm or less, 200 μm or less, 150 μm or less, or 100 μm or less. In an embodiment, a thickness of a single layer of the epidermal layer may be about 50 μm.

In the present disclosure, the method may further comprise, after Step (c-2), (c-3) forming a coating layer on the epidermal layer, containing collagen; and (c-4) forming an epidermal layer on the coating layer by three-dimensionally printing the bioink composition for an epidermal layer containing the keratinocytes isolated from skin tissue.

In the present disclosure, Steps (c-3) and (c-4) are processes for laminating the epidermal layer into multiple layers.

Steps (c-3) and (c-4) may be repeatedly performed two to four times.

In an embodiment, when Steps (c-3) and (c-4) are repeated twice, the produced skin tissue model may comprise two coating layers containing collagen and three epidermal layers.

The coating layer containing collagen may serve as an adhesive for improving adhesion between the epidermal layers.

The coating layer may comprise 0.5 to 2% collagen, preferably 0.7 to 1.5% collagen, more preferably 0.8 to 1.2% collagen, and most preferably 1% collagen layer.

The forming of the coating layer may be performed through a process of immersing in a collagen-containing solution and drying.

### Skin Tissue Model, Skin Tissue Graft Material

In another general aspect, the present disclosure provides a skin tissue model produced according to the method for producing a skin tissue model of the present disclosure.

The skin tissue model produced according to the method for producing a skin tissue model of the present disclosure may be used as a skin tissue graft material for recovering or regenerating a wound on the skin.

In the present disclosure, the skin tissue model may comprise: a dermal layer containing fibroblasts isolated from skin tissue; a first epidermal layer formed on the dermal layer, containing keratinocytes isolated from skin tissue; a first coating layer coated on the first epidermal layer, containing collagen; a second epidermal layer formed on the first coating layer, containing keratinocytes isolated from skin tissue; a second coating layer coated on the second epidermal layer, containing collagen; and a third epidermal layer formed on the second coating layer, containing keratinocytes isolated from skin tissue.

The fibroblasts isolated from the skin tissue and the keratinocytes isolated from the skin tissue may be isolated from the skin tissue isolated from a human or a patient.

In the present disclosure, the dermal layer may have a thickness of 500 to 8000 μm. For example, the lower limit thereof may be 550 μm or more, 600 μm or more, 650 μm or more, 700 μm or more, 750 μm or more, 800 μm or more, 850 μm or more, 900 μm or more, 950 μm or more, or 1000 μm or more, and the upper limit thereof may be 7500 μm or less, 7000 μm or less, 6500 μm or less, 6000 μm or less, 5500 μm or less, 5000 μm or less, 4500 μm or less, or 4000 μm or less.

In the present disclosure, a single layer of the epidermal layer may have a thickness of 15 to 500 μm. The lower limit thereof may be 20 μm or more, 25 μm or more, 30 μm or more, 35 μm or more, 40 μm or more, or 45 μm or more, and the upper limit thereof may be 450 μm or less, 400 μm or less, 350 μm or less, 300 μm or less, 250 μm or less, 200 μm or less, 150 μm or less, or 100 μm or less.

The coating layer containing collagen may serve as an adhesive for improving adhesion between the epidermal layers.

The coating layer may include 0.5 to 2% collagen, preferably 0.7 to 1.5% collagen, more preferably 0.8 to 1.2% collagen, and most preferably 1% collagen layer.

The skin tissue model according to the present disclosure may be prepared as an implantable skin tissue by isolating, processing, and proliferating a small amount of autologous or allogeneic skin collected from a cosmetically unexposed area, and subsequently applying a three-dimensional (3D) printing process.

Specifically, the present disclosure enables skin grafting with minimal donor-site morbidity by harvesting only a small amount of autologous or allogeneic skin. If necessary, allogeneic skin from which immune cells have been removed to eliminate immune responses may also be used. The removal of immune cells may be performed as needed, regardless of whether the skin is autologous or allogeneic.

Through the immune-cell removal process, immune rejection can be minimized, and a skin graft optimized to the required graft area can be fabricated using three-dimensional (3D) printing technology.

The skin tissue model according to the present disclosure can effectively minimize donor site burden and morbidity during both the harvesting and transplantation stages for the patient or donor.

The skin tissue model of the present disclosure may contribute to tissue integration and the establishment of a stable regenerative environment by enhancing physical and functional connections between the transplanted and host tissues through collagen fiber deposition and ECM remodeling, and may function as a matrix that actively promotes ECM remodeling in vivo.

### Method for Treating Skin Wound or Regenerating Skin of Skin Tissue Model or Skin Tissue Graft Material

There is provided a method for treating a skin wound or a method for regenerating the skin, the method comprising: transplanting a skin tissue model or a skin tissue transplant material produced according to the present disclosure to an individual.

The term “individual” of the present disclosure may include an animal or a human whose symptoms may be improved by transplantation of the skin tissue model or the skin tissue graft material produced according to the present disclosure, and in particular, the individual may provide the skin tissue used in the method for producing the skin tissue model of the present disclosure. By administering the skin tissue model or the skin tissue graft material produced according to the present disclosure to an individual, it is possible to effectively treat a wound or regenerate or recover the skin.

In the present disclosure, an example of the scar may be a scar in which the epidermis; dermis; subcutaneous; epidermis and dermis; dermis and subcutaneous; or epidermis, dermis, and subcutaneous layers are damaged, and preferably, it may be a full-thickness scar in which the dermis is damaged, or a full-thickness scar in which the epidermis, dermis, and subcutaneous layers are damaged.

In the present disclosure, the wound refers to an injury to a human body caused by a disorder or disease in which tissue is cut, torn, broken, burned, or traumatized, or causes such an injury. The wound may be an open wound or a closed wound with no open surface. An example of the wound may be an open wound on the skin. An example of the wound may be a wound in which the epidermis; dermis; epidermis and dermis; or the epidermis, dermis, and subcutaneous fat layer are damaged, and preferably a full-thickness skin defect with damage reaching the dermis.

### Advantageous Effects

The present disclosure provides a method for producing a skin tissue model by consistently isolating and proliferating cells from skin tissue of a patient and using bioink with optimal components and contents for forming skin tissue, thereby providing a skin tissue model which is patient-specific, minimizes side effects caused by transplantation, and solves a slow recovery problem, limitations of a donor site, etc.

In addition, the bioink composition for a dermal layer or an epidermal layer according to the present disclosure may be employed as a graft material through three-dimensional printing tailored to the shape and size of a graft site.

Further, the bioink composition for an epidermal layer and the dermal layer including the cells isolated from the skin tissue of a patient of the present disclosure may have excellent cell viability and survival maintenance ability.

### DESCRIPTION OF DRAWINGS

FIG. 1 shows the sequential production process of a skin tissue model of the present disclosure.

FIG. 2 shows results of the number of cells isolated from a tissue and the cell area for subculture.

FIG. 3 shows the results of DAPI staining and H&E staining analysis of the dermis decellularized extracellular matrix produced according to the present disclosure and native dermis tissue.

FIG. 4 shows the results of DNA and protein analysis of the dermis decellularized extracellular matrix produced according to the present disclosure and native dermis tissue.

FIG. 5 is SEM images of the dermis decellularized extracellular matrix produced according to the present disclosure and native dermis tissue.

FIG. 6 shows the results of analyzing collagen 1/collagen 3 and collagen1-alpha1/collagen3-alpha3 of the dermis decellularized extracellular matrix according to the present disclosure and native dermal tissue.

FIG. 7 shows the results of proteomics analysis of the dermis decellularized extracellular matrix according to the present disclosure.

FIG. 8 shows the identification through immunostaining by comparing fibroblasts and keratinocytes isolated from skin tissues according to the present disclosure with purchased cells.

FIG. 9 shows the analysis results of physical properties such as viscosity, compressive modulus, and elastic modulus depending on the composition ratio of components of the bioink composition for a dermal layer according to the present disclosure.

FIG. 10 shows stress relaxation at a complex elastic modulus and a strain of 10% depending on a frequency of the bioink composition for a dermal layer according to the present disclosure.

FIG. 11 shows the printability of the bioink composition for a dermal layer according to the present disclosure.

FIG. 12 shows the cell viability and cell proliferation rate of the bioink composition for a dermal layer according to the present disclosure.

FIG. 13 shows the analysis results of physical properties such as viscosity, compressive modulus, and elastic modulus depending on the composition ratio of components of the bioink composition for an epidermal layer according to the present disclosure.

FIG. 14 shows the printability of the bioink composition for an epidermal layer according to the present disclosure.

FIG. 15 shows the cell viability and cell proliferation rate of the bioink composition for an epidermal layer according to the present disclosure.

FIG. 16 shows the expression of markers in the epidermal layer depending on whether keratin-alginate ink is present in the bioink for the epidermal layer according to the present disclosure.

FIG. 17 shows the expression of a close adhesion marker depending on the formation of a barrier on the epidermis produced with the bioink for an epidermal layer according to the present disclosure.

FIG. 18 shows the verification of transverse epithelial electrical resistance (TEER) for the epidermis produced with the bioink for the epidermal layer according to the present disclosure.

FIG. 19 shows the expression of a collagen marker depending on the presence or absence of the dermis decellularized extracellular matrix in the bioink for the dermal layer according to the present disclosure.

FIG. 20 shows the expression of alpha-SMA contributing to cell migration (wound regeneration) and wound contraction by comparing the bioink for the dermal layer according to the present disclosure with the ink without a decellularization substrate and the ink containing an acellular dermal matrix.

FIG. 21 shows the thickness and height confirmed by a cell tracker after printing bioink for the dermal layer and bioink for the epidermal layer according to the present disclosure.

FIG. 22 is a polarizing microscope image of a skin model produced with the bioink for the dermal layer and the bioink for the epidermal layer according to the present disclosure and a skin model composed only of a synthetic polymer, measured after picrosirius red staining.

FIG. 23 shows images and analysis results of vascularization using the 14th-day conditioned media of the skin model produced with the bioink for the dermal layer and the bioink for the epidermal layer according to the present disclosure and the skin model composed only of the synthetic polymer.

FIG. 24 shows the results of cytokine experiments for the skin model of the present disclosure, a model using acellular dermis, and the skin model composed only of the synthetic polymers.

FIG. 25 shows the results of polarization of M0 macrophages (THP-1) in the conditioned media of the skin model of the present disclosure (pddECM+KA) and the control group (G10A1, without pddECM+KA) verified by fluorescence immunostaining of CD206 and CD163 markers.

FIG. 26 shows the results of M0 macrophages (THP-1) in the conditioned media of the skin model of the present disclosure (pddECM+KA) and the control group (G10A1, without pddECM+KA) verified by fluorescence immunostaining of CD80 marker.

FIG. 27 shows the image results of subcutaneous transplantation of the skin model (pddECM+KA) of the present disclosure and the control group (G10A1, without pddECM+KA) according to the presence or absence of cells.

FIG. 28 shows histopathological evaluation results of major organs after subcutaneous transplantation of the skin model (pddECM+KA) of the present disclosure and the control group (G10A1, without pddECM+KA) according to the presence or absence of cells.

FIG. 29 shows the results of wound induction evaluation to verify the skin regeneration effects of the skin model (pddECM+KA) of the present disclosure and the control group (G10A1, without pddECM+KA).

### BEST MODE

Hereinafter, embodiments of the present disclosure will be described in detail so that those skilled in the art can easily implement the present disclosure. However, the present disclosure may be implemented in various different forms and is not limited to the embodiments described herein.

### Preparation Example 1. Preparation of Dermis Decellularized Extracellular Matrix (dermis dECM)

### (1) Preparation of Dermis Decellularized Extracellular Matrix

Decellularization and disinfection of tissues were performed using 1 mM EDTA+0.25% Trypsin, Triton X-100, 10 mM MgCl₂, DNase (Sigma-Aldrich), 0.1% peracetic acid. The decellularized tissue was frozen at −90° C. and then treated through lyophilization and centrifugal grinder. Next, a pre-hydrogel was prepared by dissolving the decellularized tissue in a 0.5 M acetic acid solution containing pepsin (from porcine gastric mucosa) at about 10% of the tissue weight. Then, titration was performed at pH 7.4 using 10M NaOH.

### (2) Confirmation of Cell Nucleus Removal of Prepared Dermis Decellularized Extracellular Matrix

Cell nucleus staining and hematoxylin and eosin staining were used to confirm whether cells within the tissue were well removed through decellularization. Specifically, native dermis and the prepared dermis dECM were prepared as samples. The samples were fixed using 4% PFA and dehydrated using sucrose. After freezing at −90° C., the samples were embedded in O.C.T Compound (Leica) to prepare a cryosection mold. Then, the samples were sectioned at a thickness of 20 um using Cryostat, dried for one day, and fixed to the slide.

In the case of DAPI, the samples were prepared by washing three times with PBS, treating with a DAPI solution diluted 1:3500 (0.4 μL in 1 mL) for 10 minutes, and then washing three times again with PBS. In the case of H&E, the samples were washed with tertiary distilled water, treated with hematoxylin for 5 minutes, and then washed with water to remove any remaining hematoxylin. Thereafter, the samples were treated with bluing reagent for 10 seconds, treated with eosin for 2-3 minutes, followed by ethanol treatment for each concentration to conduct staining. After DAPI and H&E staining, the samples were treated with 2-3 drops of mounting medium and sealed with a cover glass for fixation. All stainings were analyzed using a slide scanner (Leica), and the results are shown in FIG. 3.

Referring to FIG. 3, DAPI was expressed before decellularization, whereas expression was significantly reduced after decellularization. In addition, it was confirmed that the cell nucleus stained with hematoxylin was observed before H&E staining, but significantly decreased after decellularization.

### (3) Comparison of DNA Content Between Prepared Dermis Decellularized Extracellular Substrate and Native Dermal Tissue

The extracellular matrix (dECM) of the decellularized dermis and the patient's dermal tissue were lyophilized for 48 hours to prepare samples. The experiment was performed using GeneJET Genomic DNA purification Kit and Picogreen assay kit. The prepared sample (5 mg) was put into a 2 mL tube, digestion solution and proteinase K solution were added thereto, and the mixture was placed at 60° C. overnight for 16 hours. In addition, RNase A solution was added, and the mixture was treated with vortex, and then incubated at room temperature for 10 minutes. Thereafter, Lysis solution was added and pipetted for 15 seconds, followed by the addition of 50% ethanol and vortexing of the mixture. Next, DNA purification column and collection tube were prepared, and centrifugation was performed for 1 minute at 6,000×g. The underlayer liquid was discarded, followed by treatment with Wash Buffer I and centrifugation at 8,000×g for 1 minute. Then, the underlayer liquid was discarded, followed by treatment with Wash Buffer II and centrifugation at 12,000×g for 3 minutes. Then, the underlayer liquid was discarded, the the remaining sample was transferred to a new 1.5 mL tube, treated with Elution Buffer for 2 minutes, and centrifuged at 8,000×g for 1 minute. DNA was immediately used and placed at −20° C. upon storage.

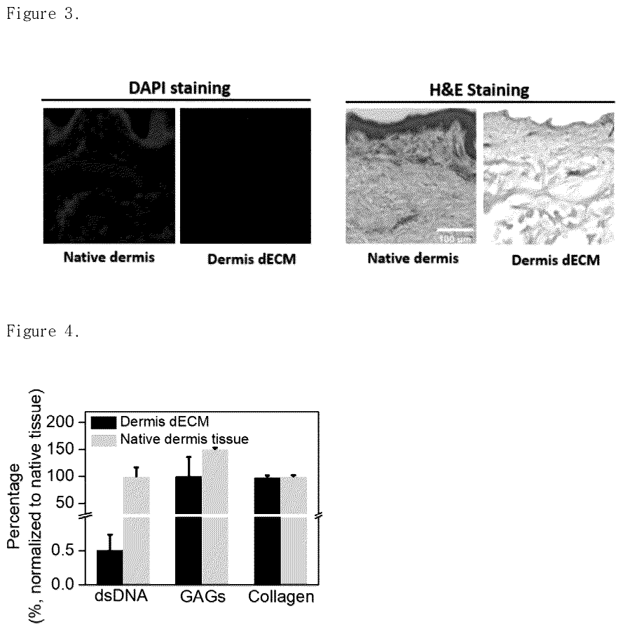

The isolated DNA was subjected to DNA content analysis through Quant-iT Picogreen dsDNA Reagent Kit. Native dermal tissue DNA samples and dECM DNA samples were treated with TE buffer (diluted 1×TE) to adjust the final dose to 100 μL. Then, 100 μL of Quant-iT Picogreen dsDNA reagent diluted 200-fold was poured to adjust the final volume to 200 μL. The samples were pipetted and incubated at room temperature for 5 minutes. Fluorescence intensity (excitation˜480 nm, emission˜520 nm) was measured using a microplate reader. DNA content was obtained through standard curve listed in the user guide, and normalized to the DNA sample from native dermal tissue, and the results are as shown in FIG. 4.

Referring to FIG. 4, it was found that the DNA content from the native dermal tissue was 1322.270047 ng/mL±218.9871664 ng/mL, and the DNA content from the dermis decellularized extracellular matrix was 6.690851863 ng/mL±3.090820569 ng/mL. As described above, it was confirmed that the DNA content was significantly reduced, indicating that decellularization treatment proceeded efficiently.

### (4) Analysis of Components and Structural Preservation of Prepared Dermis Decellularized Extracellular Matrix

Whether ECM components were preserved was analyzed through GAG and collagen measurements. Samples in all experiments were used in a finely cut state after lyophilization treatment. Then, the digestion process was performed with a papain solution. Papain from Carica papaya (25 mg/mL, 76216-50MG) was prepared in a solution containing Na₂-EDTA (03690-100ML), sodium phosphate dibasic (Na₂HPO₄) (795410-100G), sodium phosphate monobasic (NaH₂PO₄) (S2554-100G), and cysteine HCl (C121800-5G) at pH 6.6, and dissolved to 25 mg/mL solution for use.

In addition, the total amount of GAG was quantified by measuring sulfated GAG using Sulfated Glycosaminoglycan Quantification Kit (AMSbio, 280560-N). The remaining collagen was estimated using Picosens™ Hydroxyproline Assay Kit (Colorimetric) (BM-HYP-100) according to the manufacturer's protocol, and the analysis results are shown in FIG. 4.

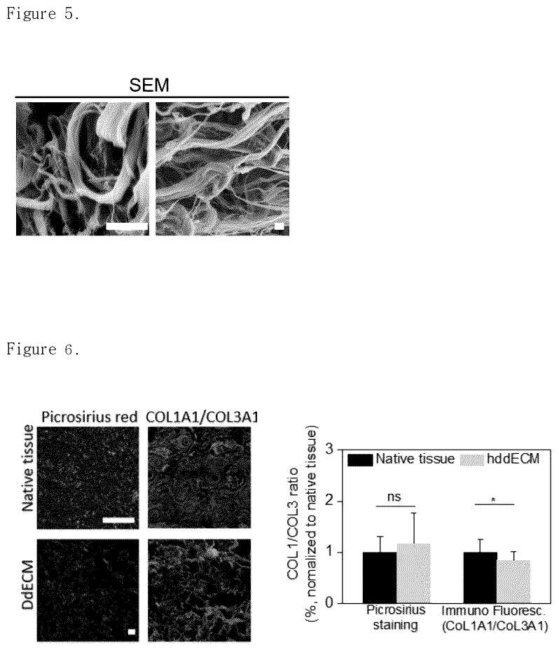

SEM analysis was conducted to verify whether the complex microstructure was maintained similar to the existing native dermis tissue even after decellularization treatment, and the analysis result is as shown in FIG. 5.

Referring to FIG. 4, the changes in GAG and collagen contents before and after decellularization treatment were assessed, confirming that GAG and collagen contents were maintained.

In addition, it was confirmed from FIG. 5 that the thin fibers and thick fibers formed a complex microstructure shape even after the decellularization treatment.

(5) Analysis of Collagen 1/Collagen 3, Collagen1-Alpha1/Collagen3-Alpha1 between Prepared Dermis Decellularized Extracellular Matrix and Native Dermal Tissue

The dermis decellularized extracellular matrix prepared according to Preparation Example 1 and the native dermal tissue were analyzed in terms of collagen 1/collagen 3, collagen1-alpha1/collagen3-alpha1. Specifically, in order to confirm the arrangement of collagen1 and collagen3, the samples were stained with picrosirius red solution (ab246832) for 1 hour. Thereafter, washing was performed twice with 0.1% acetic acid. The stained samples were measured with a polarizing microscope. RGB colors were extracted with Image J, and collagen 1/collagen 3 was analyzed by the area ratio between red (collagen1) and green (collagen3). The results are shown in FIG. 6. In addition, collagen1-alpha1 and collagen3-alpha1 were confirmed by immunofluorescence staining. Immunofluorescence staining was performed in the same manner as that performed in Example 2. Further, the expression intensity ratio between collagen1-alpha1 and collagen3-alpha1 was analyzed, and the results are shown in FIG. 6.

As shown in FIG. 6, the result of Picrosirius red staining was not significantly different, while immunofluorescence staining was reduced by about 0.85-fold.

### (6) Proteomic Analysis of Decellularized Extracellular Matrix

Proteomic analysis of the decellularized extracellular matrix (dECM) of Preparation Example 1 was performed. A specific experimental method is as follows.

### 1.1. Bravo FASP Digestion

1. For each well in the filter plate, each sample was loaded from the filter plate through vacuuming, followed by FASP digestion.
2. FASP was performed on a Bravo auto liquid handler machine (Agilent), which was operated using the VWork software on a connected PC.
3. 500 mM TCEP (Final concentration=5 mM TCEP) was added, followed by shaking at 33° C. at 300 rpm for 1 minute, and incubation for 30 minutes to reduce the protein.
4. Vacuuming was performed until 20 to 30 μL of solution remained. (30 to 1 hour, target pressure=350 to 400 mbar)
5. 500 mM IAA (final concentration=50 mM IAA) was added, followed by shaking at 25° C. at 500 rpm for 1 minute, and incubation in the dark for 1 hour to alkylate the protein.
6. Vacuuming was performed in the same manner as before.
7. Vacuuming was performed with 8 M urea buffer 100 μL (×3).
8. Vacuuming was performed with 50 mM ABC 100 μL (×3).
9. Trypsin was dissolved in 50 mM ABC and added in a concentration such that trypsin to protein was 1 to 50 ratio. The corresponding solution was digested with the peptide by shaking at 300 rpm for 1 minute at 37° C. and then overnight incubation.
10. After replacing with a new collector plate, vacuuming was performed.
11. After complete elution, 125 μL of 50 mM ABC was added, and vacuuming was performed (×2).
12. The eluted solutions were combined and dried using Speed-Vac.

### 1.2. Desalting

1) Desalting was performed using C18 Micro Spin-Column to remove salt.
2) Column Preparation
   75 μL of 80% ACN (in 0.1% TFA) was added.
   75 μL of 99.9% DIW (in 0.1% TFA) was added.
   75 μL of 99.9% DIW, 0.1% TFA was added (×2).
3) Sample Preparation
   75 μL of 99.9% DIW (in 0.1% TFA) was added.
4) Sample Loading
5) 75 μL of 99.9% DIW (in 0.1% TFA) was added to remove unwanted substances.
6) Peptide Elution
   75 μL of 80% ACN (in 0.1% TFA) was added (×2).
   75 μL of 100% ACN was added.
   75 μL of 80% ACN (in 0.1% TFA) was added.
7) After desalting, the samples were dried using Speed-vac to remove all solutions, and stored at −20° C. until analysis.

1.3. LC-MS/MS Analysis

| Parameters | Conditions | | | | | | | | | | |
|---|---|---|---|---|---|---|---|---|---|---|---|
| Trapping | C₁₈, 3 μm, 100 Å, 75 μm × 2 cm | | | | | | | | | | |
| Column | | | | | | | | | | | |
| Analytical | PepMap ™ RSLC C₁₈ | | | | | | | | | | |
| Column | 2 μm, 100 Å, 75 μm × 50 cm | | | | | | | | | | |
| Mobile Phase | A: Water with 0.1% formic acid | | | | | | | | | | |
| | B: 80% ACN with 0.1% formic acid | | | | | | | | | | |
| | Time (min) | 0 | 0.1 | 1 | 6 | 130 | 160 | 160.1 | 170 | 170.1 | 185 |
| Gradient | Solvent B (%) | 4 | 4 | 8 | 8 | 30 | 54 | 96 | 96 | 4 | 4 |
| Column Flow | 300 nL/min | | | | | | | | | | |
| Rate | | | | | | | | | | | |

| | |
|---|---|
| Mass Range | 400~2000 m/z |

### 1.4. Data Analysis

1) For data analysis, LC-MS/MS data from each sample were analyzed using Proteome Discover.
2) The Homo sapiens database from Uniprot was used.
3) Data analysis was performed by the addition of modifications.

| | Fixed | | | |
|---|---|---|---|---|
| Parameters | modification | Variable modification | | |
| Modification | Carbamido- | Oxidation | Carbamylation | Acetylation |
| Modification | methylation | Methionine | Protein | Protein |
| site | Cysteine | | N-terminal | N-terminal |

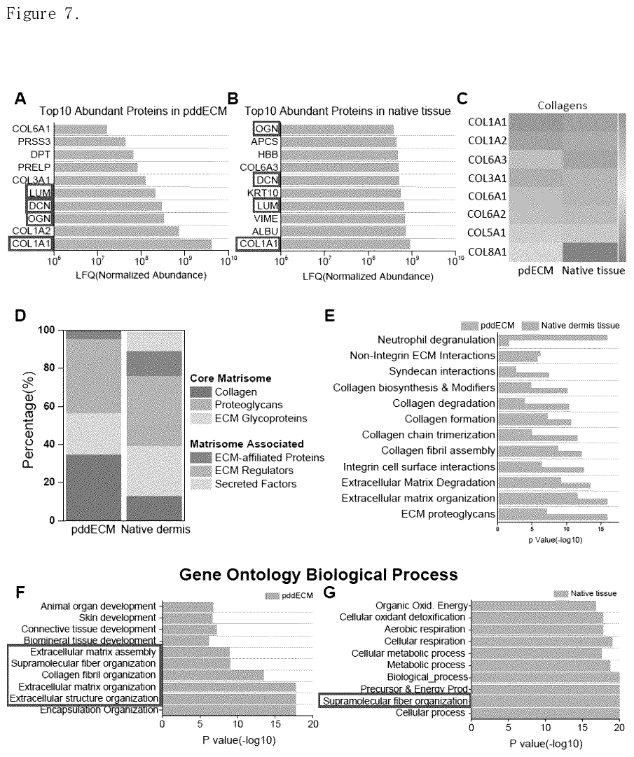

In addition, based on the data on the type and content of protein, the data were quantified using pantherdb.org and matrisomeDB sites, and the results are shown in FIG. 7.

The pddECM hydrogel of the present invention retains an ECM protein composition similar to that of native dermis tissue even after decellularization. Analysis of the top 10 common proteins revealed that COL1A1, osteoglycin (OGN), decorin (DCN), lumican (LUM), and the like were consistently present, indicating that core ECM components of the tissue were preserved during the decellularization process. In the pddECM, key fibrous and matrix regulatory proteins such as collagen type I (COL1A1, COL1A2), type III (COL3A1), and type VI (COL6A1), along with DCN, LUM, DPT, PRSS3, PRELP, were maintained at high concentrations, enabling both mechanical support of the tissue and cellular activation and regeneration induction functions.

The composition of collagen was maintained consistently before and after decellularization, with a slight increase in types I and III, and stable preservation of type VI collagen. This indicates that the pddECM reflects the native dermal basement fiber network and provides a structure suitable for cellular support and remodeling at the wound site.

Furthermore, proteins within the pddECM are gradually degraded by in vivo enzymes such as MMPs, and the ECM-derived peptides released in this process can perform complex physiological functions such as promoting cell migration, modulating inflammation, angiogenesis, and inhibiting fibrosis. DCN is involved in the regulation of fibrosis by binding to TGF-β, LUM and OGN contribute to collagen fiber alignment and stabilization of fibrous tissue. PRELP stabilizes the interaction between ECM and collagen, thereby suppressing matrix degradation, and DPT is involved in cell adhesion and matrix formation processes.

According to matrisome classification analysis, the pddECM exhibits high contents of collagens, proteoglycans, and ECM glycoproteins, while the composition of ECM-affiliated proteins and regulators was lower than that of native dermis. This indicates that it has been refined into a form with enhanced tissue support and cell-matrix interaction functions.

According to Reactome and Gene Ontology analyses, the pddECM preserves skin ECM-specific pathways such as ECM organization, collagen fibril assembly, ECM degradation, and integrin-mediated interaction, and maintains biological functions related to structural formation such as “extracellular matrix assembly” and “supramolecular fiber organization.” Accordingly, it has been confirmed that the pddECM hydrogel of the present invention is not merely a simple mixture of proteins, but rather a complex biomaterial that reflects the functional composition of skin extracellular matrix (ECM), and serves as a high-performance regenerative scaffold capable of simultaneously providing structural support and delivering bioactivity at the implantation site.

### Preparation Example 2. Fibroblast Culture Medium

A medium containing 10% fetal bovine serum, 4.5 mg/mL D-glucose, 5 mL of penicillin/streptomycin (10,000 UI/mL), 0.584 mg/mL L-glutamine, 0.03 mg/mL glycine, and 1 uL/mL fibroblast growth factor but not containing pyruvate and hydroxyethylpiperazine ethanesulfonic acid was prepared.

### Preparation Example 3. Keratinocyte Culture Medium

KBM medium containing 5 ml of penicillin, streptomycin, 0.5 mL glutamine, 2 ml of human epidermal growth factor, 0.5 ml of recombinant human insulin, 0.5 ml of hydrocortisone, 0.5 ml of gentamicin/amphotericin-B, 0.25 ml of epinephrine, and 0.5 ml of transferrin based on 500 mL of keratinocyte basal medium was prepared.

### Example 1. Production of Skin Tissue Model

### Example 1-1. Cell Isolation from Patient Skin Tissue

### (1) Preparation of Skin Tissues and Isolation of Epidermis and Dermis

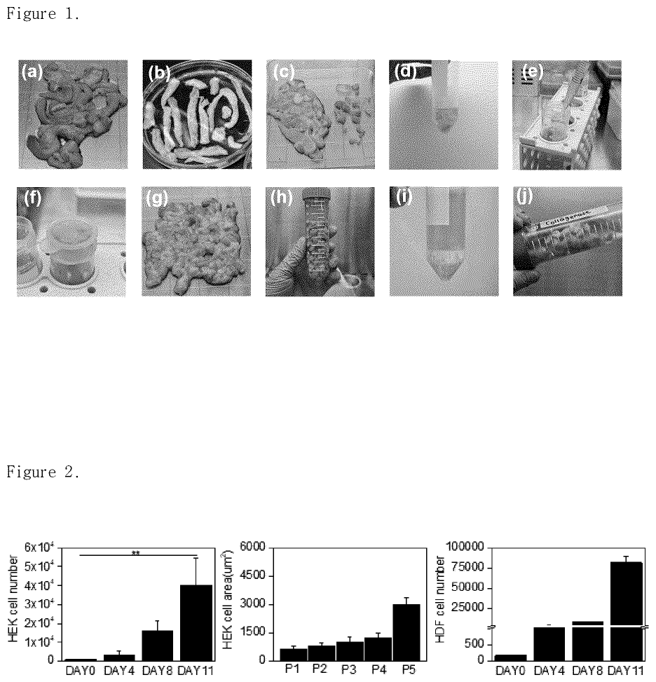

First, skin tissue isolated from a patient was washed three or more times with Phosphate Buffered Saline (PBS), and blood, burnt tissue, dissociated tissue, hair, and fat were removed using blade. The skin tissue was minimally exposed to room temperature, and a phosphate-buffered saline solution was continuously added to the tissue so as not to dry (FIG. 1(a)).

Then, the tissue was cut into 2-3 mm-wide pieces, placed with the epidermis facing down, and then treated at 4° C. for 15-18 hours in 2.4 UI/mL of dispase-II solution, which was added until the epidermis was submerged (FIG. 1(b)). Thereafter, the tissue treated with the dispase-II solution was isolated into epidermis and dermis by forceps, and washed with PBS to remove remaining dispase-II (FIG. 1(c)).

Here, the dispase-II solution was prepared at 2.4 UI/mL by adding the dispase-II solute (Gibco) to phosphate buffered saline.

(2) Isolation of Keratinocytes from Isolated Epidermis

The isolated epidermis obtained in Step (1) was washed with PBS to remove the dispase solution, and then treated with 1 mL of 0.05% trypsin-ethylenediaminetetraacetic acid (EDTA) (trypsin-EDTA) per 1 cm² of tissue at 37° C. for 30 minutes. The sample was carefully shaken every 5 minutes to increase cell isolation efficiency (FIG. 1(d)).

The cell culture medium was then added in an amount approximately twice that of Trypsin-EDTA to neutralize Trypsin-EDTA. The neutralized solution was carefully pipetted to isolate the cells (FIG. 1(e)).

Next, the neutralized tissue was filtered through a cell strainer fitted into a tube. The filtered tissue was placed in a new tube, the keratinocyte culture medium of Preparation Example 3 was poured in a small amount, and then cells remaining on the epidermis were isolated using a vortex mixer. The tissue was once again filtered using a cell strainer, and then the experiment was carried out using only the obtained solution. The supernatant was removed by centrifugation, and the suspended matter was dispersed in the keratinocyte culture medium of Preparation Example 3 and transferred to a cell culture flask. After 3 days, the medium was replaced with the keratinocyte culture medium of Preparation Example 3 (FIG. 1(f)).

Thereafter, the culture plate was washed twice with phosphate-buffered saline (PBS). The culture plate was enzymatically treated with 0.025% trypsin-EDTA at 37° C., neutralized with cell culture medium, and a keratinocyte pellet was prepared, which was then used to prepare bioink.

The results of analyzing the number of keratinocytes (HEK cells) isolated according to the above method and the cell area for subculture are shown in FIG. 2.

(3) Isolation of Dermal Fibroblasts from Dermis

Meanwhile, the isolated dermis obtained in Step (1) was washed once with PBS, and then the fat layer present deeply in the dermis was completely removed and cut finely (FIG. 1(g)).

Thereafter, the pre-heated collagenase-I solution was added at 1.5 mL per 1 cm² of tissue, and the mixture was pipetted. Then, the tissue was treated at 37° C. for 30-60 minutes and gently shaken every 5 minutes (FIG. 1(h)). The collagenase-I solution was prepared using a keratinocyte culture medium (KBM) without the keratinocyte growth factor of Preparation Example 3. It was prepared by dissolving collagenase-I solute in a medium at 625 UI/mL. In addition, the fibroblast culture medium was prepared to include 100 μl of fibroblast growth factor per 1 mL at a concentration of 0.07% based on the fibroblast culture medium of Preparation Example 2.

Next, the treated dermis was pipetted, and collagenase-I was removed using a centrifuge (FIG. 1(i)). To completely remove collagenase-I, the process of adding fibroblast culture medium of Preparation Example 2, pipetting, and then removing the supernatant using a centrifuge was repeated three times.

Then, the cell culture medium was added to the tissue washed three times with fibroblast culture medium of Preparation Example 2, and pipetted. The tissue was transferred to a culture flask, and removed after 3 days (FIG. 1(j)).

Thereafter, the culture plate was washed twice with phosphate-buffered saline (PBS). The culture plate was enzymatically treated with 0.05% trypsin-EDTA at 37° C., neutralized with cell culture medium, and fibroblast pellet was prepared, which was then used to prepare bioink.

The analysis results of the number of fibroblasts (HDF cells) isolated according to the method above are shown in FIG. 2.

### Example 1-2. Preparation of Bioink Composition for Dermal Layer Containing Isolated Cells

### (1) Preparation of Bioink Composition

The bioink composition for a dermal layer contained the dermis dECM of Preparation Example 1, dermal fibroblasts isolated according to Example 1-1, gelatin methacryloyl (GelMA) and alginate.

First, bioinks were prepared by mixing GelMA and alginate with the compositions shown in Table 1 below.

TABLE 1
Bioink

| | | |
|---|---|---|
| | Gelatin Methacryloyl | |
| Classification | (GelMA) | Alginate |

| | | | |
|---|---|---|---|
| Composition 1 | 10 | w/v % | 1 w/v % |
| Composition 2 | 10 | w/v % | 2 w/v % |
| Composition 3 | 7.5 | w/v % | 2 w/v % |

In addition, bioink compositions were prepared by mixing the dermis decellularized extracellular matrix of Preparation Composition 1 with the bioink of Composition 1 at a ratio (v/v) of 1:2, 1:3, and 1:4.

### (2) Preparation of Bioink Composition for Dermal Layer

The bioink composition prepared above was filtered with a sterile filter, stored at a temperature of 43° C. before use, and placed at 37° C. for 30 minutes before mixing with cells.

The fibroblast pellet prepared in Example 1-1 was added to the heated bioink composition and pipetted to prepare a bioink composition for a dermal layer. The composition was carefully pipetted for about 1 minute, as pipetting had to be completed before gelation occurred at room temperature. In this case, the bioink composition for a dermal layer contained about 5,000,000-8,000,000 (5×10⁶ to 8×10⁶ cells/mL) fibroblasts per mL of ink. The prepared bioink composition was placed in a sterilized piston cartridge and placed at 4° C. for 10 minutes to undergo physical gelation.

### Example 1-3. Preparation of Bioink Composition for Epidermal Layer Containing Isolated Cells

### (1) Preparation of Bioink Composition

A bioink composition for an epidermal layer contained keratinocytes isolated according to Example 1-1, gelatin methacryloyl (GelMA), and keratin-alginate.

First, bioink compositions were prepared by mixing GelMA and keratin-alginate with the composition shown in Table 2 below.

TABLE 2

| | Bioink Composition | | |
|---|---|---|---|
| | | Gelatin Methacryloyl | Keratin- |
| Classification | | (GelMA) | alginate |

| | | | | |
|---|---|---|---|---|
| Composition 1 | 10 | w/v % | 1 | w/v % |
| Composition 2 | 10 | w/v % | 1.5 | w/v % |
| Composition 3 | 10 | w/v % | 2 | w/v % |
| Composition 4 | 7 | w/v % | 1 | w/v % |
| Composition 5 | 7 | w/v % | 1.5 | w/v % |
| Composition 6 | 7 | w/v % | 2 | w/v % |
| Composition 7 | 5 | w/v % | 1 | w/v % |
| Composition 8 | 5 | w/v % | 1.5 | w/v % |
| Composition 9 | 5 | w/v % | 2 | w/v % |

Ink was prepared by adding gelatin methacryloyl (GelMA), a keratin-alginate solute, and 0.5 w/v % 2-hydroxy-4′-(2-hydroxyethoxy)-2-methylpropiophenone to a phosphate buffer solution, followed by reaction at 80° C. for 10 minutes, and mixing with a vortex mixer. For even dispersion, the process of treating at 80° C. for 10 minutes and then mixing with a vortex mixer was repeated 4-5 times.

The keratinocyte pellet prepared in Example 1-1 was added to the heated bioink composition and pipetted to prepare a bioink composition for a dermal layer. The composition was carefully pipetted for about 1 minute, as pipetting had to be completed before gelation occurred at room temperature. Here, the bioink for the epidermal layer contained about 2,000,000 (2×10⁶ cells/mL) keratinocytes per mL of ink. The prepared bioink was placed in a sterilized piston cartridge and placed at 4° C. for 10 minutes to undergo physical gelation.

### Example 1-4. Production of Skin Tissue Model Including Patient-Derived Cells and Patient-Derived Decellularized Ink by Bioprinting

Prior to printing the bioink for the dermal layer prepared in Example 1-2, the printer bed temperature was set to 12° C., the following settings were made and printing was performed: the printer bed temperature was 12° C., the printer head temperature was 22-25° C., the pressure was 80 kPa-100 kPa, and the speed was 2 mm/s, and the nozzle was 27G with a diameter of 0.2 mm.

The bioink for the dermal layer prepared in Example 1-2 was printed on a transwell insert to print a dermal layer having a height of 200 μm. The bioink for the epidermal layer of Example 1-3 was used to print on the printed dermal layer at a height of 50 μm, and left to adhere for about 2 hours.

At this time, in the step of 3D bio-printing the bioink to produce a skin tissue model, 1% collagen coating was performed on one layer of the epidermis in order to increase adhesion between the epidermis, and the bioink was immersed in a coating solution for about 30 minutes. Then, the epidermal layer was printed again thereon at 50 μm. The epidermal layer was printed three times, and collagen coating was performed twice between the three layers.

### Example 2. Characteristics and Morphological Analysis of Cells Isolated from Tissues

In order to confirm whether the cells isolated from the tissue according to Example 1 were fibroblasts and keratinocytes, changes in the model according to proliferation were observed through immunostaining.

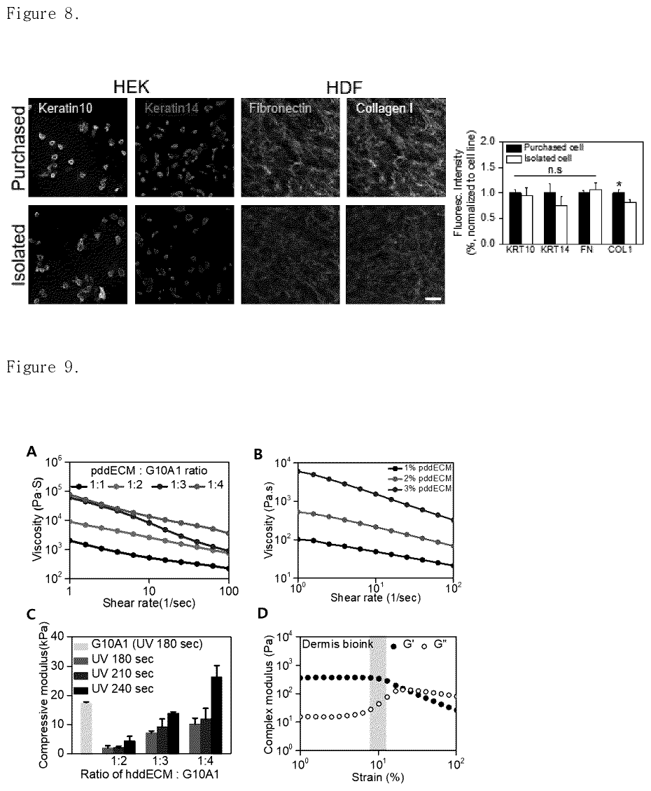

Specifically, the isolated cells cultured in 2D for 7 days were stained with antibodies. After removing the culture medium contained in the culture flask, the cells were washed twice with PBS. The cells were fixed with 4% paraformaldehyde at room temperature for 20 minutes, washed twice with PBS, and permeabilization of the cells was increased at room temperature for 30 minutes using PBS in which 0.1% Triton X-100 was dissolved. Thereafter, the cells were washed once with PBS, and blocked at room temperature for 15 minutes using a solution in which 1% bovine serum albumin (BSA) was dissolved in PBS. Subsequently, the first antibody was diluted in a ratio of 1:250 (4 μL in 1 mL 1% BSA solution) and reacted overnight (about 18 hours) at 4° C. After the primary antibody treatment, washing was performed twice with PBS. 2% Goat serum (1 mL in 50 mL 1% BSA solution) was prepared and treated at room temperature for 15 minutes. Next, a second antibody was diluted in a ratio of 1:250 (4 μL in 1 mL 2% Goat Serum in 1% BSA solution), DAPI was added in a ratio of 1:3500 (0.4 μL in 1 mL), and the mixture was treated at 37° C. for 20 minutes in a state in which light was blocked. Thereafter, staining was completed by washing twice with PBS, and the results are shown in FIG. 8.

Referring to FIG. 8, human epidermal keratinocyte (HEK) had a flat and rounded shape, and it was confirmed that the cells isolated through Keratin14 and Keratin10 staining were keratinocytes. In addition, as keratinocytes proliferated, melanocytes were rarely found, proving that keratinocytes in a healthy state were isolated. The melanocytes could be removed through differential trypsinization using trypsin-EDTA treatment at room temperature for 1-2 minutes. It was confirmed that human dermal fibroblasts (HDF) had spindle- or star-shaped, long, thin, and extended protrusions as they proliferated, and cells isolated from the tissue were confirmed to be fibroblasts through fibronectin and collagen staining.

In addition, cell identity was confirmed by cross-verifying protein expression intensity by comparing keratinocytes and fibroblasts isolated from patients with commercial cells. There was no significant difference in Keratin10, Keratin14 and Fibronectin expression, but Collagen I expression decreased about 0.87-fold compared to commercial fibroblasts.

### Example 3. Characteristic Analysis of Bioink for Dermal Layer

### (1) Analysis of Physical Properties According to Ink Mixing Ratio of Bioink for Dermal Layer

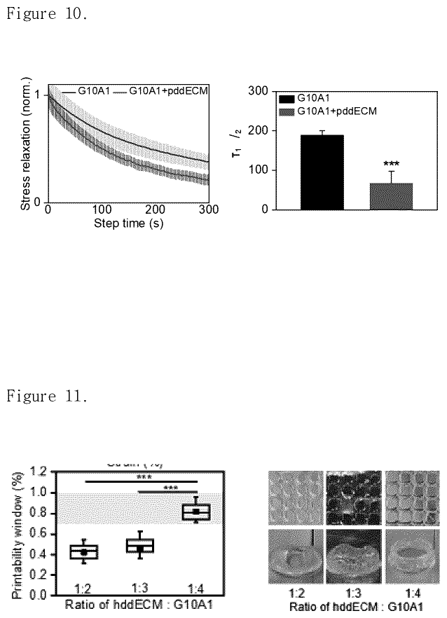

The physical properties of the dermal layer bioink of Example 1-2 depending on the mixing ratio of the components were confirmed. Specifically, the bioink was measured at room temperature of 25° C. using HR20 Rheometer and parallel plate (8 mm diameter). In the viscosity-shear rate test, the shear rate was measured in the range of 1 to 100 s⁻¹. The complex modulus was measured at 25° C. at strains from 1.0% to 100%. The compressive strength was measured at a rate of 33.33 um/s, the sample was prepared and analyzed with a diameter of 8 mm and a thickness of 1 mm, and the analysis results are shown in FIG. 9. In addition, the results of stress relaxation behavior and stress relaxation half-life (T₁/2) over time under the condition of strain of 10% are shown in FIG. 10.

Referring to FIG. 9, when the mixing ratio of decellularized extracellular matrix and bioink composed of gelatin methacryloyl (GelMA) and alginate was 1:4, the bioink had a uniform and rigid shape. With respect to the compression modulus value, as the ratio between GelMA and alginate increased, and as the UV crosslinking time increased, the compression modulus value increased.

In addition, referring to FIG. 9A, a viscosity value of dECM, in which the viscosity decreases as the shear rate increases, was shown. GelMA and Alginate were added to improve the physical properties, and the improved physical properties are shown in FIGS. 9B and 9C.

Further, as shown in FIG. 9B, the change in viscosity of bioink for dermal layer according to shear rate was confirmed depending on the mixing ratio of ink, and the decrease in viscosity with increasing shear rate indicated shear-thinning behavior, confirming that the bioink for dermal layer prepared according to Example 1-2 had properties that allow for extrusion printing.

In addition, when the mixing ratio of the decellularized extracellular matrix and the bioink composed of gelatin methacryloyl (GelMA) and alginate was 1:4 (1% dECM), it was found that the viscosity was relatively high compared to other ratios.

Moreover, referring to FIG. 9D, when strain was 10% or less, the elastic modulus (G′) was higher than the viscous modulus (G″).

In addition, referring to FIG. 10, under a strain of 10%, the G10A1+dECM1 condition showed lower composite elastic moduli (G′ and G″) than that of G10A1 across the entire frequency range. Further, in the stress relaxation test, the G10A1+dECM1 condition showed a faster stress relaxation rate, and the stress relaxation half-life (T₁/2) decreased by about 0.35-fold compared to G10A1.

### (2) Analysis of Printability of Bioink for Dermal Layer

The possibility of printing the bioink for the dermal layer prepared according to Example 1-2 was analyzed. Specifically, for the printability test, printing was performed using 27G needle on a BIO X printer of Cellink. The printability was analyzed using the ratio between the width that should be theoretically printed and the actual printed width, and the results are shown in FIG. 11.

Referring to FIG. 11, it was shown that the bioink for the dermal layer composed of dECM:GelMA and alginate-containing bioink in a ratio of 1:4 had high printability.

### (3) Analysis of Cell Viability of Bioink for Dermal Layer

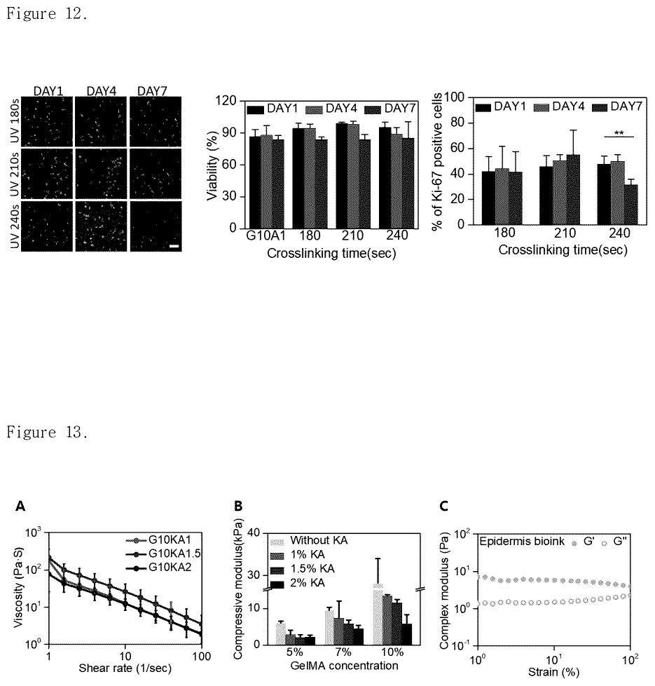

The cell viability of the bioink for dermal layer prepared according to Example 1-2 was analyzed by Live/Dead staining and Ki-67 staining, and specifically, for Live/Dead staining, the sample was washed twice with PBS for 30 minutes each. Then, a solution was prepared with 5 μm of Calcein AM (4 mM in anhydrous DMSO) and 20 μm of Ethidium homodimer-1 (2 mM in DMSO/H₂O 1:4 (v/v)) in 10 mL PBS, and the solution was poured enough to submerge the sample, followed by incubation at 37° C. for 1 hour while blocking light. The treated sample was washed twice with PBS for 1 hour. Ki-67 staining was performed in the same manner as the immunostaining method of Example 2, the primary antibody was treated with Ki-67, and the secondary antibody suitable for ki-67 and dapi were stained together, and the analysis results are shown in FIG. 12.

Referring to FIG. 12, as a result of analyzing cell viability according to UV crosslinking time in Live/Dead staining, it was found that cell viability was maintained at 83.67% (±2.59%, 180 sec), 83.95% (±4.67%, 210 sec), and 85.32% (±15.61%, 240 sec) until Day 7. In addition, Ki-67 staining confirmed that cell proliferation rates were maintained or slightly increased on Day 7 under 180 and 210-second conditions.

Considering the physical properties, cell viability, cell proliferation rate and printability of the bioink composition for a dermal layer of the present disclosure, it was confirmed that the formation of the dermal layer under a condition in which the bioink composition for a dermal layer composed of the ratio of dECM:GelMA & Alginate=1:4 was subjected to UV crosslinking time for 210 seconds was the optimum condition

### Example 4. Characteristic Analysis of Bioink for Epidermal Layer

### (1) Analysis of Physical Properties of Bioink for Epidermal Layer

The swelling ratio, compression modulus, and viscosity of the bioink for the epidermal layer of Example 1-3 depending on the content ratio of gelatin methacryloyl (GelMA) and keratin-alginate (KA) were analyzed. Specifically, the bioink was measured at room temperature of 25° C. using a HR20 Rheometer and a parallel plate (8 mm diameter). In the viscosity-shear rate test, the shear rate was measured in the range of 1 to 100 s⁻¹. The compressive modulus was measured using samples with a diameter of 8 mm and a thickness of 1.5 mm, and analyzed at strains from 1.0% to 10%. The results are shown in FIG. 13.

Referring to FIG. 13A, it was found that the bioink for the epidermal layer composed of the ratio of 10% GelMA+1.5% KA had the highest viscosity.

In addition, referring to FIG. 13B, when the content of GelMA was 10 w/v %, the bioink for the epidermal layer exhibited a high compressive modulus. In addition, the bioink for the epidermal layer composed of 10% GelMA+2% KA showed the lowest swelling ratio, confirming relatively structurally stable characteristics compared to other content ratios.

Moreover, referring to FIG. 13C, when strain was 10% or less, the elastic modulus (G′) was higher than the viscous modulus (G″).

### (2) Analysis of Printability of Bioink for Epidermal Layer

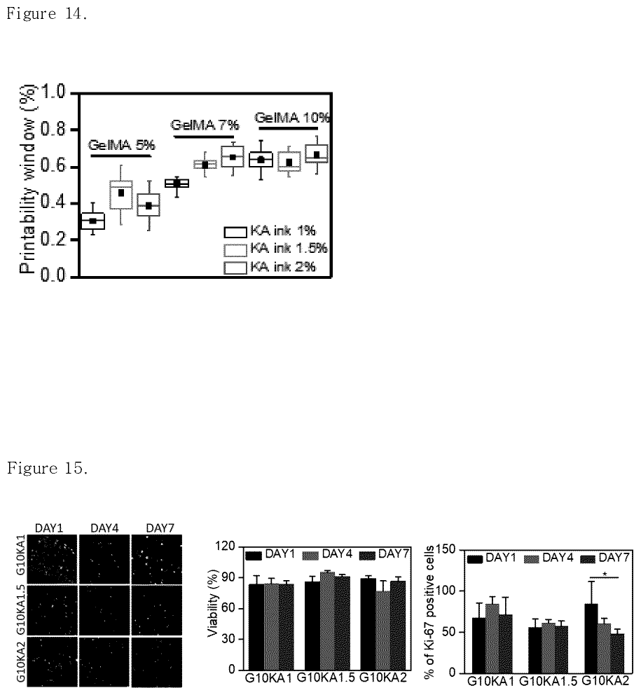

The possibility of printing the bioink for the epidermal layer prepared according to Example 1-3 was analyzed. Specifically, for the printability test, printing was performed using 27G needle on a BIO X printer of Cellink. The printing suitability was analyzed using the ratio between the width that should be theoretically printed and the actual printed width, and the results are shown in FIG. 14.

Referring to FIG. 14, it was shown that the bioink for the epidermal layer based on GelMA 10% had high printability.

### (3) Analysis of Cell Viability of Bioink for Epidermal Layer

The cell viability depending on the content ratio of gelatin methacryloyl (GelMA) and keratin-alginate (KA) of the bioink for the epidermal layer prepared according to Example 1-3 was analyzed using Live/Dead staining and Ki-67 staining, and the results are shown in FIG. 15.

As shown in FIG. 15, the bioinks for the epidermal layer composed of a ratio of GelMA 10%+KA 1%, GelMA 10%+KA 1.5%, and GelMA 10%+KA 2% maintained cell viability until Day 7, similar to Day 1. In addition, it was found that the bioink composition for an epidermal layer composed of the ratio of GelMA 10%+KA 1% and GelMA 10%+KA 1.5% maintained the Ki-67 positive cells value even on Day 7.

As a result of the analysis, cell viability, assessed by Live/Dead staining, showed no difference for each condition over time, and maintained between 80-90% in all conditions. The analysis of Ki-67 positive showed that the Ki-67 marker was the most activated in the bioink composition for an epidermal layer with a composition of GelMA10%+KA1% when viewed on Day 7, indicating the highest proliferation rate.

Therefore, considering the physical properties, cell viability, cell proliferation rate, and printability of the bioink composition for an epidermal layer of the present disclosure, it was confirmed that the bioink composition for an epidermal layer composed of 10% GelMA and 1% to 2% KA was suitable for forming an epidermal layer for forming a skin tissue model.

### Example 5. Confirmation of Marker Expression and Barrier Formation in Bioink for Epidermal Layer and Bioink for Dermal Layer According to Present Disclosure

### (1) Confirmation of Marker Expression of Epidermal Layer Depending on Inclusion of Keratin-Alginate in Bioink for Epidermal Layer

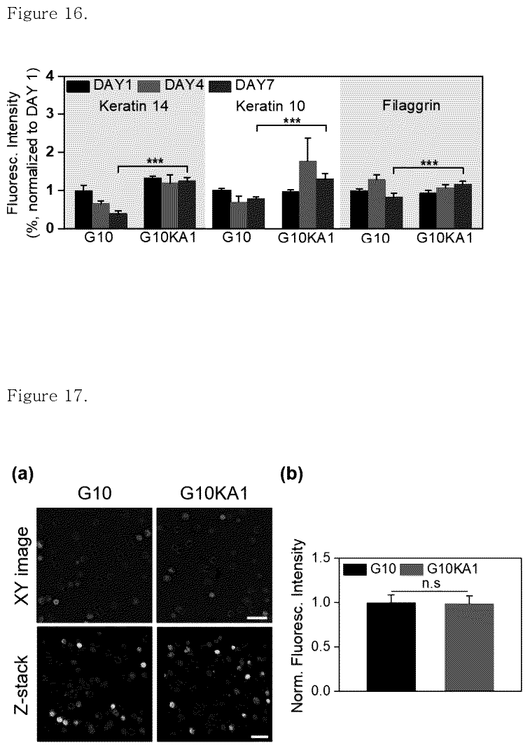

Immunofluorescence staining was performed to confirm how the inclusion of keratin-alginate as a bioink component for the epidermal layer affected marker expression, and the results are shown in FIG. 16.

Referring to FIG. 16, in the expression of Keratin 14, the G10 condition without keratin-alginate showed a decreased expression on Day 7 (˜0.40-fold change compared to Day 1), whereas the G10KA1 condition, the bioink for the epidermal layer containing keratin-alginate, generally maintained the expression (˜0.94-fold change compared to Day 1).

In addition, in the expression of Keratin 10, the G10 condition showed a slightly decreased expression on Day 7 (˜0.79-fold change compared to Day 1), whereas the G10KA1 condition showed a marked increase in expression (˜1.34-fold change compared to Day 1).

Further, in the expression of 7-day FIL, G10 showed a decreased expression (˜0.82-fold change compared to Day 1), whereas G10KA1 conditions showed an increased expression (˜1.25-fold change compared to Day 7). In other words, G10KA1 showed gradual expression, and confirmed gradual progression of keratinization until Day 7.

In order to confirm whether the epidermis produced with bioink for the epidermal layer was closely adhered, immunofluorescence staining was performed to confirm the expression of the Zonula Occludens (ZO)-1 marker, and the result is as shown in FIG. 17.

Referring to FIG. 17, it was confirmed that there was no difference in expression between the skin model (pddECM+KA) of the present study and the control group (G10A1, without pddECM+KA).

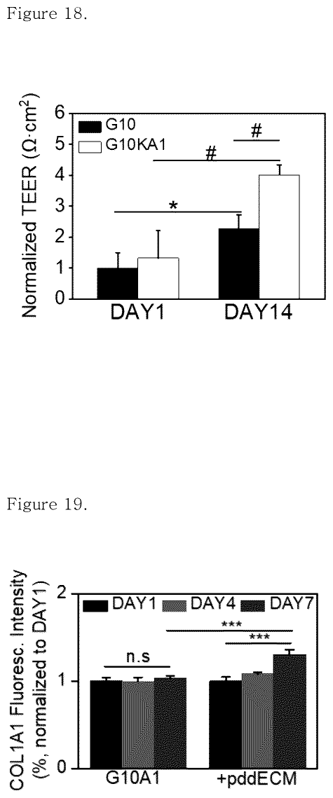

Transverse epithelial electrical resistance (TEER) was measured in order to verify whether a barrier was formed on the epidermis produced by using bioink for the epidermal layer, and the results are shown in FIG. 18.

Referring to FIG. 18, G10 on Day 14 showed a 2.26-fold increase in TEER compared to G10 on Day 1, and G10KA1 on Day 14 showed a significant 3.03-fold increase in TEER compared to G10KA1 on Day 1.

### (2) Confirmation of Marker Expression of Dermal Layer Depending on Inclusion of Decellularized Extracellular Matrix in Bioink for Dermal Layer

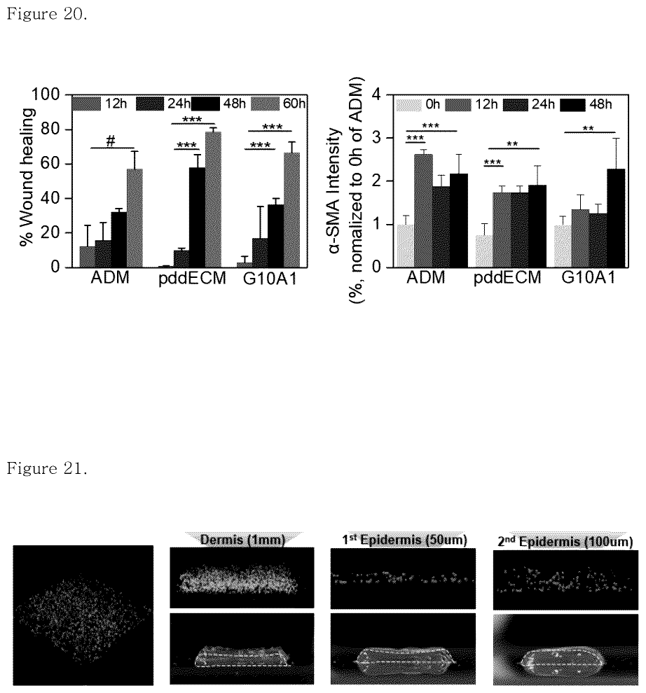

Expression of a collagen marker in the dermal layer was confirmed depending on whether or not the decellularized extracellular matrix as a bioink component for the dermal layer was included. Specifically, the expression of COL1A1 in ink containing pddECM (G10A1dECM1) and ink without pddECM (G10A1) was confirmed, and analysis was conducted by setting Day1 of ink without pddECM as 1, and the results are as shown in FIG. 19.

Referring to FIG. 19, the ink without pddECM showed a tendency to maintain intensity over time (˜1.03-fold change from Day1 to Day7). However, the ink containing dECM showed an increase over time (˜1.30-fold change from day1 to day7). When comparing the COL1A1 expression values on Day 7 between the two ink conditions, the pddECM-containing condition showed a 1.26-fold higher value. This increase means improvement of collagen type 1 synthesis capability (p<0.005).

### (3) Analysis of Wound Healing and Alpha-SMA Expression of Bioink for Dermal Layer

Wound healing and alpha-SMA expression were confirmed using the bioink for the dermal layer (pddECM) according to the present disclosure, the ink without decellularized extracellular matrix (G10A1), and the ink containing an acellular dermal matrix (ADM). The three inks were heated at 37° C. for 30 minutes and then thinly coated on 24 well plate. Each ink was UV crosslinked for 210 seconds and dried for one day. The experiment was performed by seeding of 40,000 HDF per 1 cm² and culturing HDF for 3-4 days. A linear scratch was made with a 200 ul pipette tip, and wound healing was observed over time, and the alpha-SMA at that time was examined. The result is as shown in FIG. 20.

As shown in FIG. 20, the wound healing rate (%) was 78.34% (±2.51), which was significantly high in the bioink for the dermal layer according to the present disclosure.

In addition, as a result of α-SMA Intensity analysis, which is a marker expressed in cells contributing to contraction, it was confirmed that the expression value of the bioink condition for the dermal layer according to the present disclosure at 48 hours was not significantly different from those of other control groups.

Collectively, these results demonstrated that the proliferation of fibroblasts had a more effective wound healing effect than myofibroblasts, which contribute to contraction.

### Example 6. Characteristic Analysis of 3D Printed Skin Tissue Model Produced According to the Present Disclosure

The characteristics of the skin tissue model (pddECM) produced according to Example 1-4, the skin model (G10A1) composed only of synthetic polymers, and an acellular dermal-based skin model without an extracellular matrix were analyzed.

The skin model (G10A1) composed only of the synthetic polymer was a skin model produced in the same manner as in Example 1-4 by using synthetic polymers, gelatin methacrylate (GelMA) and alginate, without including the bioink composition for a dermal layer and the bioink composition for an epidermal layer of the present disclosure. Further, the acellular dermal-based skin model (ADM) is a skin model produced in the same manner as in Example 1-4 of the present disclosure, but does not include an extracellular matrix.

The thickness and height of the skin tissue model produced according to the present disclosure were analyzed using a cell tracker, and as a result, it was confirmed that the dermal layer was 1 mm, with the first epidermal layer measuring 50 μm, and the second epidermal layer measuring 100 μm (FIG. 21).

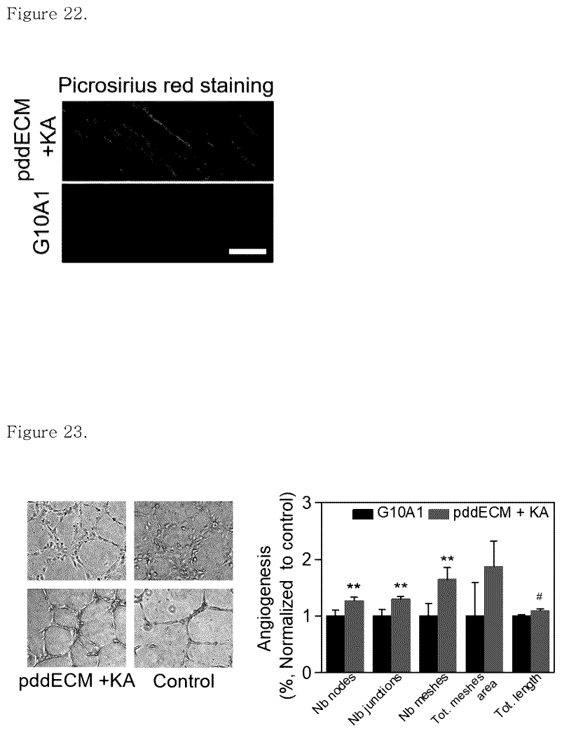

In addition, Picrosirius red staining analysis for the skin tissue model (pddECM) prepared according to the present disclosure and the skin model (G10A1) composed only of synthetic polymer revealed that collagen production and accumulation were confirmed in the skin tissue model (pddECM) of the present disclosure, but not observed in the skin model (G10A1) composed only of synthetic polymer (FIG. 22).

Picrosirius Red staining revealed collagen fiber deposition in the pddECM-based skin tissue model, indicating that ECM synthesis and remodeling occurred concurrently. Localized deposition of collagen fibers represents an early stage of matrix reconstruction by fibroblasts, forming a new ECM structure in the damaged tissue, thereby providing a foundation for cell adhesion, migration, and differentiation. This process is essential in forming physical and functional connections between the grafted and host tissues and contributes to tissue integration and the establishment of a stable regenerative environment. Accordingly, the collagen deposition induced by pddECM plays a role beyond simple structural support, functioning as a bioactive matrix that actively promotes ECM remodeling in vivo.

Furthermore, the vascularization of the skin tissue model (pddECM) produced according to the present disclosure and the skin model (G10A1) composed only of synthetic polymers was confirmed. 24-Well plate of well was thinly coated with 150 μL of Geltrex and treated at 37° C. for 30 minutes, and Human Umbilical Vein Endothelial Cells (HUVECs) were seeded at 90,000 cells/well. Then, the 14th-day conditioned media of the skin model of the present disclosure and the control group were diluted 1:1 with HUVEC media (growth factors and serum not included), and the cells were cultured. Vascularization was confirmed for 4 hours, 8 hours, and 12 hours, and the analysis was performed by “Angiogenesis Analyzer” plugin for ImageJ software, and the results are shown in FIG. 23.

As shown in FIG. 23, the skin model of the present disclosure showed an increase in node, junction, and mesh compared to G10A1 (˜1.27, ˜1.30, and ˜1.64-fold changes compared to G10A1 at 4 h). These results reflect network expansion due to the generation of new blood vessels and suggest the possibility of promoting wound healing by enhancing vascularization during skin transplantation.

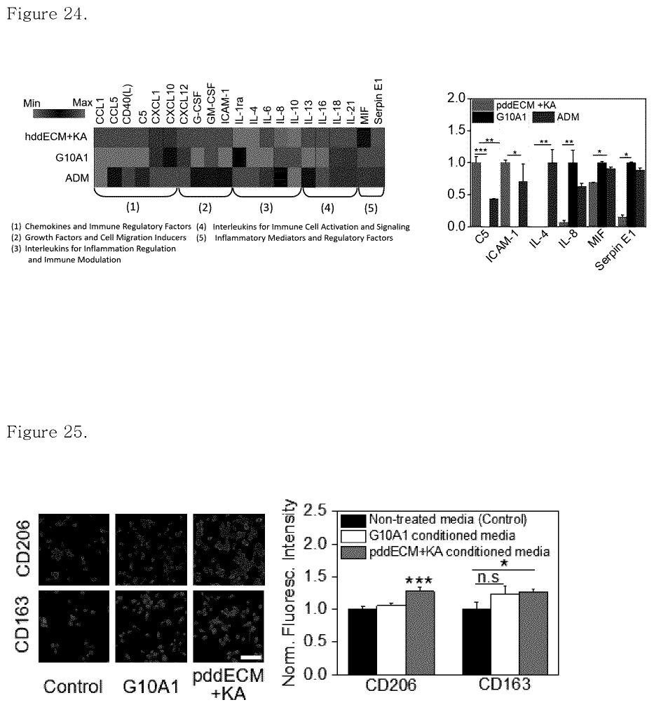

In addition, cytokine analysis was performed using the skin tissue model (pddECM) prepared according to the present disclosure, the acellular dermal-based skin model (ADM), and the skin model (G10A1) composed only of synthetic polymers, and the results are shown in FIG. 24.

As shown in FIG. 24, the up-regulated expression of Complement Component C5 (C5) and Intercellular Adhesion Molecule 1 (ICAM-1) was shown in a model with pddECM and KA inks. Low expression was observed in Interleukin-4 (IL-4), Interleukin-8 (IL-8), and endothelial plasminogen activator inhibitor (Serpin E1). Expression of C5 may accelerate wound healing, promote collagen accumulation, and enhance hemostatic effects. Expression of ICAM-1 contributes to wound healing, keratinocyte migration to the center of the wound, and tissue formation. On the other hand, the non-expression of IL-4 may result in a lack of activation of macrophages, a delay in collagen synthesis, etc., but IL-4 is also known as a major factor causing fibrosis. In addition, IL-8 is involved in wound closure and angiogenesis, but may cause inflammatory skin diseases and oxidative stress. It was also found that overexpression of Serpin E1 could lead to fibrosis and tissue hardening.

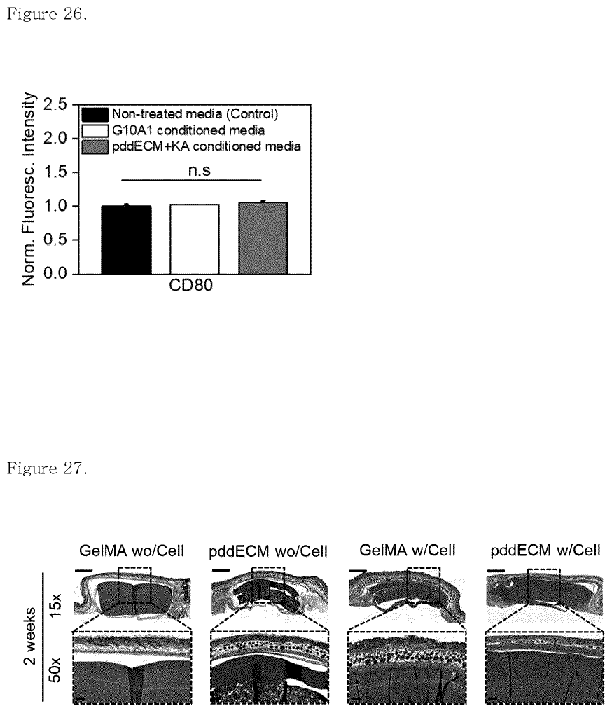

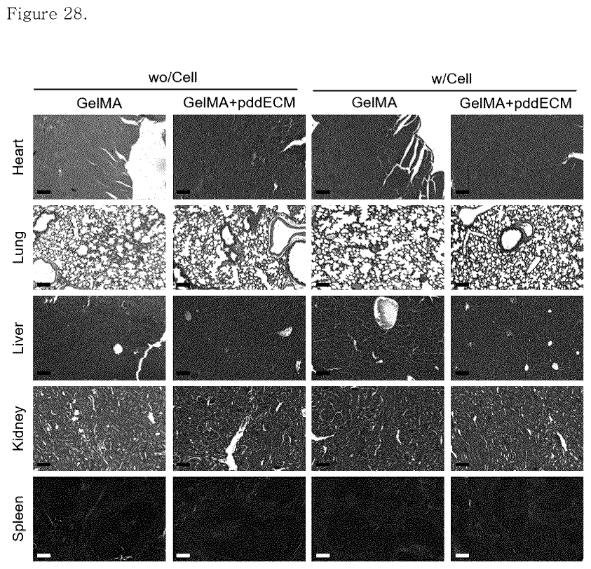

In addition, the polarization tendency of M0 macrophages (THP-1) was evaluated using the conditioned media of the skin tissue model (pddECM+KA) produced according to the present disclosure and the control group (G10A1 without pddECM+KA). To this end, immunofluorescence staining was performed on the M1 marker (CD80) and the M2 markers (CD206, CD163), and the results are shown in FIGS. 25 and 26.

As shown in FIG. 25, the control group (G10A1 without pddECM+KA) and the skin tissue model (pddECM+KA) produced according to the present disclosure showed a 1.06-fold and 1.27-fold increase in CD206 expression, respectively, compared to the non-treated group (i.e., group not treated with conditioned media). The expression of CD163 was also increased by about 1.23-fold and 1.27-fold under the conditions of G10A1 and pddECM+KA, respectively, but this was not a phenomenon specific to pddECM+KA and was also observed in G10A1.

As shown in FIG. 26, it was confirmed that CD80 showed no significant difference compared to the non-treated group (i.e., group not treated with conditioned media).

### Example 7. In Vivo Grafting Experiment of 3D Printed Skin Tissue Model Produced According to the Present Disclosure

The skin tissue model (pddECM) produced according to Example 1-4 and the skin model (G10A1) composed only of synthetic polymers were divided into 4 groups (GelMA without (wo)/Cell, GelMA+pddECM without (wo)/Cell, GelMA w/Cell, and GelMA+pddECM w/Cell) depending on the presence or absence of cells, and were subcutaneously transplanted into 7-week-old male C57Bl/6 mice, and then wound regeneration in nude mice was confirmed.

According to FIG. 27, as a result of the subcutaneous transplantation, the fibrous capsules around the graft material were formed thicker in the group containing cells (GelMA w/Cell, and pddECM w/Cell) than in the group not containing cells (GelMA wo/Cell, and pddECM wo/Cell). In all groups, subcutaneously grafted materials showed no apparent foreign body response in the surrounding tissues, and showed a degradation pattern starting from the peripheral areas.

According to FIG. 28, a toxic reaction was confirmed through histopathological evaluation of major organs (heart, lung, liver, kidney, and spleen) for 2 weeks. No significant toxic reactions were observed in the heart, lungs, and kidneys, but mild to moderate reactive hepatocytes were observed in the liver, and the group containing cells also showed a slight proliferation of the splenic white pulp. These results indicate that the transplantation of scaffolds and cells did not cause local or systemic toxicity in the transplanted animals.

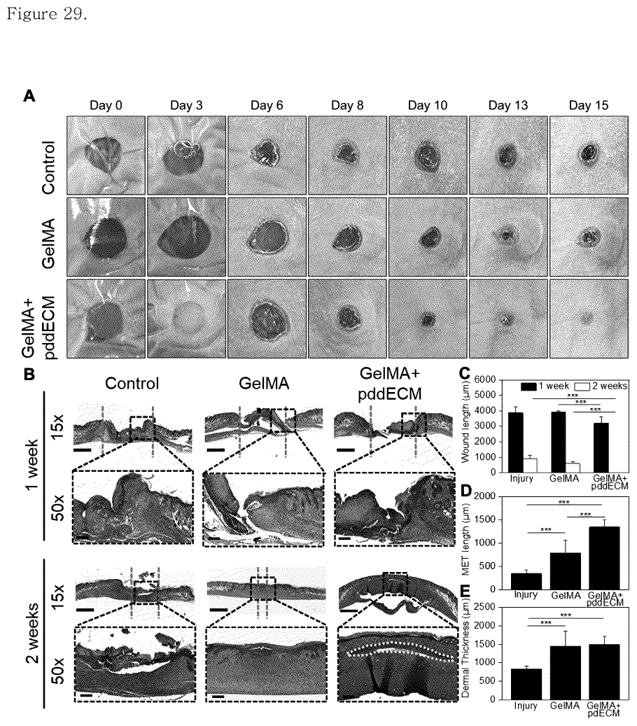

According to FIG. 29, in order to evaluate whether the transplantation of the model according to the present disclosure improved skin regeneration, 6 mm circular wound was made on nude mouse, a graft was inserted, and then the regeneration characteristics were analyzed for 2 weeks.

In addition, according to FIG. 29A, as a result of visual inspection, there was no clear change in wound size until Day 7, but from Day 13, wound healing was improved in the scaffold-grafted group (GelMA, GelMA+pddECM). At week 2, incomplete epidermis and congestions of the epidermal region were partially observed in the control group (G10A1 without pddECM+KA).

Furthermore, as indicated by the dashed yellow line in FIG. 29B, the GelMA+pddECM graft exhibited epidermal regeneration similar to that of the stratum basale.

In addition, according to FIG. 29C, quantitative analysis results confirmed a statistically significant difference in wound length between the GelMA+pddECM group, which showed complete healing at week 2, and the remaining groups.

In particular, according to FIG. 29D, the MET length was about 2.3-fold longer in the GelMA group and about 3.9-fold longer in the GelMA+pddECM group, compared to the control group. Further, the GelMA+pddECM group was also about 1.7-fold longer in MET length than the GelMA group, indicating that the addition of pddECM enhanced epidermal regeneration.

According to FIG. 29E, both the GelMA and GelMA+pddECM groups were significantly increased for the dermal thickness at week 1 compared to the non-grafted group, and no significant difference was observed between the GelMA and GelMA+pddECM groups.

The present disclosure has been described through the examples as described above. Those skilled in the art to which the present disclosure pertains will understand that the present disclosure may be implemented in other specific forms without changing the technical spirit or essential features thereof. Therefore, it should be understood that the above-described embodiments are exemplary and not restrictive in all aspects. The scope of the present disclosure is indicated by the claims to be described later rather than the detailed description, and it should be interpreted that all changes or modifications derived from the meaning and scope of the claims and the equivalent concept are included in the scope of the present disclosure.

1. A method for producing a skin tissue model, comprising:
(a) isolating and culturing fibroblasts and keratinocytes from skin tissue;
(b) preparing a bioink composition for a dermal layer and a bioink composition for an epidermal layer, the bioink composition containing the isolated cells; and
(c) producing a skin tissue model using the bioink composition for a dermal layer and the bioink composition for an epidermal layer.

2. The method of claim 1, wherein Step (a) comprises:
(a-1) isolating epidermis and dermis from skin tissue using a solution containing Dispase-II;
(a-2) isolating keratinocytes from the isolated epidermis using a solution containing trypsin-ethylenediaminetetraacetic acid (EDTA) (trypsin-EDTA), and culturing the isolated keratinocytes in a keratinocyte culture medium; and
(a-3) isolating the fibroblasts from the dermis using a solution containing Collagenase-I and culturing the isolated fibroblasts in a fibroblast culture medium.

3. The method of claim 2, wherein the keratinocyte culture medium is a keratinocyte basal medium (KBM) comprising penicillin/streptomycin, glutamine, epidermal growth factor (EGF), insulin, hydrocortisone, gentamicin/amphotericin-B, epinephrine, and transferrin.

4. The method of claim 2, wherein the fibroblast culture medium comprises fetal bovine serum, D-glucose, penicillin/streptomycin, L-glutamine, glycine, and fibroblast growth factor.

5. The method of claim 1, wherein the bioink composition for a dermal layer of Step (b) comprises:
fibroblasts isolated from the skin tissue;
dermis decellularized extracellular matrix (dermis dECM); and
a bioink containing gelatin methacryloyl (GelMA) and alginate.

6. The method of claim 5, wherein the bioink containing gelatin methacryloyl (GelMA) and alginate comprises 3 to 15 w/v % gelatin methacryloyl (GelMA) and 0.5 to 4 w/v % alginate.

7. The method of claim 5, wherein the bioink composition for a dermal layer is prepared by mixing the dermis dECM and the bioink containing gelatin methacryloyl (GelMA) and alginate in a volume ratio of 1:1 to 1:8.

8. The method of claim 5, wherein the bioink composition for a dermal layer comprises fibroblasts at a concentration of 1×10⁶ to 1×10⁷ cells/ml.

9. The method of claim 5, wherein the bioink composition for a dermal layer comprises the dermis dECM at a concentration of 0.5 to 2 w/v %.

10. The method of claim 5, wherein the dermis dECM comprises at least one selected from the group consisting of collagens, proteoglycans, ECM glycoproteins, ECM-affiliated proteins, and ECM regulators.

11. The method of claim 1, wherein the bioink composition for an epidermal layer of Step (b) comprises:
keratinocytes isolated from the skin tissue;
gelatin methacryloyl (GelMA); and
keratin-alginate.

12. The method of claim 11, wherein the bioink composition for an epidermal layer comprises 2 to 15 w/v % gelatin methacryloyl (GelMA) and 0.5 to 5 w/v % keratin-alginate.

13. The method of claim 11, wherein the bioink composition for an epidermal layer comprises keratinocytes at a concentration of 1×10⁶ to 4×10⁶ cells/ml.

14. The method of claim 1, wherein Step (c) comprises:
(c-1) forming a dermal layer by three-dimensionally printing the bioink composition for a dermal layer containing the fibroblasts isolated from skin tissue; and
(c-2) forming an epidermal layer on the dermal layer by three-dimensionally printing the bioink composition for an epidermal layer containing the keratinocytes isolated from skin tissue.

15. The method of claim 14, wherein Step (c-2) is performed 2 to 4 times, so that the 2 to 4 epidermal layers are laminated.

16. The method of claim 14, wherein a thickness of the dermal layer is 500 to 8000 μm and/or a thickness of a single layer of the epidermal layer is 15 to 500 μm.

17. The method of claim 14, further comprising, after Step (c-2),
(c-3) forming a coating layer on the epidermal layer, containing collagen; and
(c-4) forming an epidermal layer on the coating layer by three-dimensionally printing the bioink composition for an epidermal layer containing the keratinocytes isolated from skin tissue.

18. The method of claim 17, wherein Steps (c-3) and (c-4) are repeatedly performed two to four times.

19. A skin tissue model produced according to claim 1.

20. The skin tissue model of claim 19, wherein the skin tissue model comprises:
a dermal layer containing fibroblasts isolated from skin tissue;
a first epidermal layer formed on the dermal layer, containing keratinocytes isolated from skin tissue;
a first coating layer coated on the first epidermal layer, containing collagen;
a second epidermal layer formed on the first coating layer, containing keratinocytes isolated from skin tissue;
a second coating layer coated on the second epidermal layer, containing collagen; and
a third epidermal layer formed on the second coating layer, containing keratinocytes isolated from skin tissue.
